(19) United States
(12) Patent Application Publication
(10) Pub. No.: US 2011/0082532 A1
(43) Pub. Date: Apr. 7, 2011

(54) ADAPTER FOR RAPID CONNECTION OF PACEMAKER-ELECTRODE CATHETER

(75) Inventor: Vakeriy Khursenko, Kiev (UA)

(73) Assignee: CHAMED SRL, Formello, Roma (IT)

(21) Appl. No.: 12/995,645

(22) PCT Filed: Jun. 5, 2008
(86) PCT No.: PCT/IB08/52219

§ 371 Date: Dec. 1, 2010

(51) Int. Cl. A61N 1/05

(52) U.S. Cl. 607/122

(57) ABSTRACT

The present invention concerns an adapter which allows a rapid connection between a pacemaker and an electrode catheter, thus allowing the convenient performance of the necessary intraoperatory measurements. This adapter comprises a block of insulating material 1 with a metal insert for interconnection, a button with automatic return 2 for quick coupling to an electrode of the electrode catheter, and a slot 4, 5 for the entry of an interconnecting electrode 3.

### FIELD OF THE INVENTION

The present invention concerns the field of accessories for pacemakers (PM), devices already in use for some years for the electrical stimulation of the heart in patients without natural stimulation or with an insufficient or non-existent heart rate. As is known, these implants comprise a PM inserted in a special pocket formed in the patient's thorax, which PM generates the stimulating electric impulses, transmitted to the heart by means of a transvenous electrode catheter.

### STATE OF THE ART

In the first PM implant phase, the electrode catheter is inserted in the chosen vein, normally the cephalic vein, through a special opening and is then pushed along the subclavian vein, the brachiocephalic or innominate vein and the vena cava until its distal end arrives in the right ventricle of the heart, onto the endocardium of which it is suitably fixed.

The electrode catheters have a coaxial structure comprising an outer insulating sheath and an inner spiral composed of several wires of a special metal alloy with high electric conductivity, wound in helix formation, with the coils in close contact, which coils define an inner lumen.

The tubular and coaxial structure is essential because, as the spirals are very flexible on account of their nature and construction, it would not be possible to insert the electrode catheter along the above-mentioned veins; the operation is therefore performed, temporarily giving the electrode catheter a greater rigidity.

This greater rigidity is achieved by introducing into the cavity of the spiral a thin metal wire, sufficiently rigid and of the same length as the electrode catheter itself, called a mandrel, which is extracted at the end of the operation when the end of the electrode catheter has arrived in the ventricle to which it is fixed.

When this operation is completed, the electrode catheter remains in place to perform its function. However, after a certain period of time, for example some years, as is known to exerts in this field, it is necessary to replace the pacemaker. This replacement operation is performed in the operating room because patients who have been fitted with a PM do not have their hearts in such a state of health as to allow them enough time for the replacement of the PM. It is therefore necessary to use appliances that maintain the stimulation of the heart during replacement. On that occasion all the necessary controls and adjustments are made using appliances external to the patient.

The foresaid intraoperatory procedures consist of measuring the threshold values of cardiac stimulation, the circulating current (Ic), the threshold current (Is) and the safety factor Fs wherein: Fs=Ic/Is, and an electrical check-up of the electrode catheter is also made. These procedures, together with the direct connection of the PM to the implanted electrode catheter, present various logistic difficulties of execution which complicate the operations to be done.

Besides these problems there are also those of interconnection between PM, electrode catheter and the external measuring and regulating appliance.

At present pacemakers and electrode catheters have couplings with non unified dimensions and each type of PM is associated with electrode catheters with different dimensions, which makes it necessary, during the operation to implant or replace the PM, to insert the end of the electrode catheter distal from the heart in the coupling located on the PM or alternatively in terminals of the external appliance. Lastly, at the end of the replacement operation it is necessary to detach the end of the electrode catheter and fix it permanently onto the PM with the original screws. Moreover, the hermetic seal of the silicone rubber part is restored, which part prevents blood and physiological liquid penetrating inside the coupling and reaching the retaining screw, with the consequent risk of loss of current.

Clearly these adapting and restoring operations imply the screwing and unscrewing of the terminals carried out by medical personnel who are not always used to mechanical equipment. These manoeuvres unduly and harmfully prolong the operating times as well as increasing the risk that, after a series of screwing and unscrewing by hand, the screws and their seats may become stripped or damaged, making it impossible to use them.

Adapters for electrode catheters are currently on the market, but they are of the type with multiple tightening and present various of low reliability, such as:
The adapter is connected to the electrode by tightening a screw on the former which engages the spiral of the electrode catheter;
the pin of the adapter is connected to the pacemaker by tightening the screw which is a part of the coupling itself;
the connection by clamping the pin to the spiral of the adapter extension, this tightening is necessary because the alloy of which the electrode catheter spiral is made does not allow welding.

### AIM OF THE INVENTION

Based on the above-mentioned problems, the present invention therefore aims to supply a connecting or quick coupling system between measuring equipment and electrode catheter which allows intraoperatory measurements to be performed conveniently and easily. This aim is achieved by means of an adapter according to claim 1 and a respective method of use according to claim 8. The independent claims show other advantageous embodiments. The present invention achieves this by supplying an adapter which allows a rapid and easy connection between the electrode catheter and the leads connecting to the external measurement equipment, thus allowing convenient operation.

### DETAILED DESCRIPTION OF THE INVENTION

The present invention will be described in detail with reference to the figures. They show:

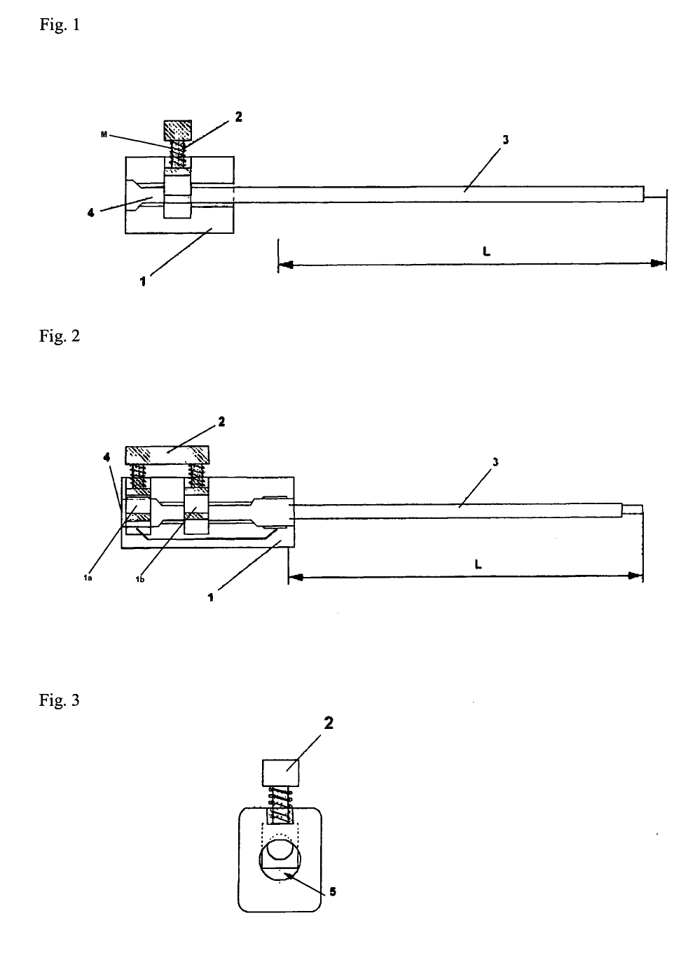

FIG. 1: rapid adapter fitted onto the end of the unipolar electrode catheter 3;

FIG. 2: rapid adapter fitted onto the end of the bipolar electrode catheter 3;

FIG. 3: front view of the rapid adapter according to the present invention.

The rapid adapter forming the object of the present invention comprises the following parts:
block of insulating material (1) with a metal insert for interconnection
button with automatic return (2) for quick coupling to an electrode or to the two electrodes of the electrode catheter
input slot (4) for an interconnecting electrode (3)

The adapter may be implemented for both a unipolar electrode catheter (FIG. 1) and a bipolar one (FIG. 2). In the second case the block 1 must be made in two sections insulated from each other, 1a and 1b, in order to guarantee a bipolar connection in both directions and to prevent any electric conduction between the two electrodes. The button with automatic return (2) acts here on both the insulated sections (1a, 1b) of the block (1). For both embodiments the button 2 is provided with a return spring M. The return of the button 2 may be made with more springs if necessary. These may be spiral springs, but also springs of another type may be used depending on the space available.

The adapter may be supplied sterile in a separate pack with the measuring equipment, or already inserted in the pack of the PM. The length L of the rapid adapter will have various measurements ranging from 20 to 80 cm but which anyway will guarantee a low ohmic resistance, so as not to distort the intraoperatory measurements.

The connection of the connecting cable of the measurement appliance to the electrode catheter 1 implanted in the patient's body is fast and is accomplished by pressing the lever 2 which opens the slot 4, 5, automatically closing it again when released, ensuring an effective and electrically valid connection.

The adapter is connected in turn to an electric cable, not illustrated in the figures with the appliance for measuring and externally stimulating the heart. The length of the connecting cable is chosen according to the distance between the measuring appliance and the patient so as to allow a convenient position for the operator who can make the measurements in a reliable way. The external measuring appliance is of course connected to the PM in a known way. For example, if the PM is of a unipolar type the return connection from the measuring appliance may be made with an additional interconnection between the plate of the PM and a plate inserted under the patient's skin. The adapter impedance must have a maximum value of 10 ohm so as not to have a significant influence on the measurements.

To achieve this result it is opportune for the adapter to be made either with a lead with several strands or with a multiwire spiral.

The adapter concerned allows a rapid interconnection between the electrode catheter and the external appliance so that the interruption of the electric supply provided by the PM to the electrode catheter, between the moment of detachment from the PM and the connection with the measuring appliance that also supplies stimulation of the heart during the period in which the PM remains detached, is very short and does not create any problem of endangering the patient's life.

Lastly, in this phase the necessary intraoperatory measurements can be easily performed. The use of this rapid adapter for attaching the measuring system is absolutely reliable because of its simplicity and does not require the use of instruments that are difficult to use in such working situations, such as small screwdrivers.

The measurements during the regulation of the PM after its replacement are made in a known way using an external programmer by means of a RF connection or with connections by means of cables.

1. An adapter for a connection between a pacemaker (PM) and an electrode catheter comprising:
a block of insulating material (1) with a metal insert for interconnection;
a button with automatic return (2) for quick coupling to an electrode of the electrode catheter; and
an entry slot (4, 5) for an interconnecting electrode (3), characterised in that the block (1) comprises two sections insulated from each other (1a, 1b) such as to guarantee a bipolar connection in both directions.

2. The adapter according to claim 1, characterised in that the button with automatic return (2) acts on both of the insulated sections (1a, 1b) of the block (1).

3. The adapter according to claim 1, characterised in that the length (L) of the interconnecting electrode (3) is between 20 and 80 cm.

4. The adapter according to claim 1, characterised in that the button with automatic return (2) is provided with a return spring (M).

5. The adapter according to claim 1, characterised in that its impedance has a maximum value of 10 Ohm.

6. The adapter according to claim 1, characterised in that it is made with a lead with several strands or with a multiwire spiral.

7. A method of rapid interconnection between a pacemaker (PM) and an electrode catheter making use of the adapter according to claim 1, comprising the phases of:
choosing a length (L) of the interconnecting electrode (3);
opening the entry slot (5) of the electrode (3) by pressing the button with return (2); and
closing the entry slot (5) of the electrode (3) by releasing the button with return (2) when the distance (L) is reached.

8. The adapter according to claim 2, characterised in that the button with automatic return (2) acts on both of the insulated sections (1a, 1b) of the block (1).

9. The adapter according to claim 2, characterised in that the length (L) of the interconnecting electrode (3) is between 20 and 80 cm.

10. The adapter according to claim 2, characterised in that the button with automatic return (2) is provided with a return spring (M).

11. The adapter according to claim 2, characterised in that its impedance has a maximum value of 10 Ohm.

12. The adapter according to claim 2, characterised in that it is made with a lead with several strands or with a multiwire spiral.

13. A method of rapid interconnection between a pacemaker (PM) and an electrode catheter making use of the adapter according to claim 2, comprising the phases of:
choosing a length (L) of the interconnecting electrode (3);
opening the entry slot (5) of the electrode (3) by pressing the button with return (2); and
closing the entry slot (5) of the electrode (3) by releasing the button with return (2) when the distance (L) is reached.

14. The adapter according to claim 3, characterised in that the button with automatic return (2) acts on both of the insulated sections (1a, 1b) of the block (1).

15. The adapter according to claim 3, characterised in that the length (L) of the interconnecting electrode (3) is between 20 and 80 cm.

16. The adapter according to claim 3, characterised in that the button with automatic return (2) is provided with a return spring (M).

17. The adapter according to claim 3, characterised in that its impedance has a maximum value of 10 Ohm.

18. The adapter according to claim 3, characterised in that it is made with a lead with several strands or with a multiwire spiral.

19. A method of rapid interconnection between a pacemaker (PM) and an electrode catheter making use of the adapter according to claim 3, comprising the phases of:
choosing a length (L) of the interconnecting electrode (3);
opening the entry slot (5) of the electrode (3) by pressing the button with return (2); and
closing the entry slot (5) of the electrode (3) by releasing the button with return (2) when the distance (L) is reached.
